(19) United States
(12) Patent Application Publication
(10) Pub. No.: US 2010/0064384 A1
(43) Pub. Date: Mar. 11, 2010

(54) METHOD FOR MODIFYING THE ATP/ADP RATIO IN CELLS

(75) Inventor: Thorsten Zank, Mannheim (DE)

Correspondence Address: CONNOLLY BOVE LODGE & HUTZ, LLP, P O BOX 2207, WILMINGTON, DE, 19899, US

(73) Assignee: Max Planck Institut fur molekulare, Potsdam (DE)

(21) Appl. No.: 12/296,340

(22) PCT Filed: Mar. 20, 2007
(86) PCT No.: PCT/EP2007/052655

§ 371 Date: Oct. 7, 2008

(30) Foreign Application Priority Data
Apr. 13, 2006 (EP) 06112638.9

(51) Int. Cl. C12N 5/10; A01H 1/00; C12N 15/82; C07H 21/04; C07K 14/415; C12N 15/63

(52) U.S. Cl. 800/278; 435/419; 435/468; 536/23.6; 530/350; 435/320.1

(57) ABSTRACT

The invention relates to a method of modifying the ATP-ADP ratio in a cell, tissue, organ, microorganism or plant by altering the hemoprotein activity in the cell, and to the use of the method.

The invention relates to a method of modifying the ATP-ADP ratio in a cell, tissue, organ, microorganism or plant by altering the hemoprotein activity in the cell, and to the use of the method.

Adenosine triphosphate (ATP) is formed from ADP (adenosine diphosphate) and energy-rich phosphate bonds both during the photosynthetic process and during respiration. These are endergonic reactions. The energy-rich ATP is hydrolyzed by means of ATPases, during which process energy is released. Since most processes in the cell are endergonic, they only become possible by coupling with a second, exergonic, reaction, which in most cases takes the form of the hydrolysis of ATP.

The hydrolysis of ATP to give ADP acts as the driving force in many biochemical processes such as, for example, active transport across membranes, biosynthesis of lipids, proteins, carbohydrates or nucleic acids.

Thus, ATP in the cell is an energy carrier which provides the energy for, ultimately, any activity of the cell or of the organism. Every organism, therefore, uses ATP as its primary energy source, ATP therefore plays a key role in cell metabolism.

On the other hand, however, ATP has a very short half-life (“lifespan”) and therefore virtually no storage capacity.

The ATP-ADP ratio is an important parameter of the energy metabolism. A high ATP-ADP ratio means an excessive energy. When the cell lacks energy, the intracellular ATP reserves are consumed, and the ATP-ADP ratio shifts towards ADP.

The expression of hemoglobin or related proteins is known from the prior art.

U.S. Pat. No. 6,372,961 discloses the expression of genes coding for hemoglobin, whereby the oxygen metabolism in plants is increased. This increased oxygen or ATP content may affect the biosynthesis in the plants. WO 98/12913 discloses a method of increasing the oxygen assimilation, which is based on the expression of hemoglobin proteins. Furthermore, this publication discloses that an increase in the production of secondary metabolites can be attributed to a simultaneous increase in the ATP concentration. Moreover, WO 00/00597 discloses that the expression of nonsymbiotic hemoglobin in cells leads to an increase of the ATP content. According to WO 99/02687 A, the expression of hemoglobin and related proteins was employed to increase the iron content in cells. In WO 2004/057946 A, a higher starch and oil yield in plants is achieved by expressing leghemoglobin.

The publication WO 2004/087755 discloses a method of increasing the stress resistance of plants and the yield obtained from them, based on the expression of plant of class two.

The expression of leghemoglobin in plant cells is furthermore known from Barata at al: (Plant Science; Vol. 155; June 2000, 193-202), where the availability of oxygen is studied.

An increase of the ATP-ADP ratio is not known from the prior art.

It is an object of the present invention to provide a method by means of which more ATP, and hence more energy, is available to the cell or the organism. In particular, it is intended that ATP is also utilized as an energy reserve, i.e. it is intended to achieve an increase in the ATP/ADP ratio.

It is a further object of the present invention to employ, in a targeted fashion, the energy thus provided for the synthesis of fatty acids, in particular alpha-linolenic acid (cis,cis,cis-9,12,15-octadecatrienoic acid).

These objects are achieved by modifying the activity of at least one hemoprotein in the method according to the invention for modifying the ATP/ADP ratio in a cell, tissue, organ, microorganism or plant.

Surprisingly, it has been found that cells, organs, tissues, microorganisms or plants with an increased ATP/ADP ratio are generated by modifying the activity of at least one hemoprotein.

The ATP/ADP ratio is understood as meaning the ratio of the concentration of ATP to the concentration of ADP. The concentrations can be determined by the customary methods known to the skilled worker, for example by means of ³¹P NMR spectroscopy in intracellular measurements, ores described hereinbelow in the examples.

Within the context of the present invention, the term cell comprises: cells, parts of plants such as organs or tissues, and intact plants and microorganisms.

Hemoproteins are proteins which are capable of binding oxygen via a prosthetic group, such as, for example, nonsymbiotic hemoglobin, myoglobin or leghemoglobin, preferably leghemoglobin and nonsymbiotic hemoglobin, especially preferably leghemoglobin.

“Activity of a hemoprotein” means the ability of the polypeptide to bind oxygen to the prosthetic group (heme). In accordance with the invention, this is understood as meaning iron(II) complexes of protoporphyrin.

An alteration in the activities of a hemoprotein in a cell means the ability to bind more or less oxygen in the cell in comparison with cells of the wild type of the same genus and species to which the methods according to the invention has not been applied under otherwise identical framework conditions (such as, for example, culture conditions, cell age and the like). The alteration, increase or reduction, preferably increase, in comparison with the wild type in this context amounts to at least 1%, 2%, 5%, 10%, preferably at least 10% or at least 20%, especially preferably at least 40% or 60%, very especially preferably at least 70% or 80%, most preferably at least 90%, 95% or more.

In one embodiment of the present invention, the ATP/ADP ratio amounts to at least 200%, preferably 300%, especially preferably at least 400% or more, based on the ATP/ADP ratio of the wild type.

The comparison is preferably carried out under analogous conditions. “Analogous conditions” means that all the framework conditions such as, for example, culture or growing conditions, assay conditions (such as buffer, temperature, substrates, concentration and the like) are kept Identical between the experiments to be compared and that the experimental combinations differ only in the activity of hemoproteins.

To modify means in accordance with the invention a de novo introduction of the activity of a polypeptide according to the invention into a cell, tissue, organ, microorganism or plant, or a reduction or, preferably, an increase of a preexisting activity of the polypeptide according to the invention. In one embodiment of the present invention, the concentration of the hemoproteins is increased.

The alteration of the activity of a hemoprotein can be achieved by modifying the structure of the proteins, by altering the stability of the hemoproteins or by altering the concentration of the hemoproteins in a cell.

A preferred variant of the present invention comprises increasing the activities of a hemoprotein, preferably of a nonsymbiotic hemoglobin or of a leghemoglobin.

It is especially preferred to increase the activity of a polypeptide which is encoded by a nucleic acid molecule comprising at least one nucleic acid molecule selected from the group consisting of:

a) nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) nucleic acid molecule which is obtained by amplifying a nucleic acid molecule from a cDNA database or from a genome database by means of the primers as shown in sequence No. 41 and 42;
f) nucleic acid molecule which codes for a polypeptide with hemoprotein activity and which hybridizes under stringent conditions with a nucleic acid molecule as shown in (a) to (C):
g) nucleic acid molecule coding for a hemoprotein which can be isolated from a DNA library under stringent hybridization conditions by using a nucleic acid molecule as shown in (a) to (c) or the subfragments thereof of at least 15 nt, preferably 20 nt, 30 nt, 50 nt, 100 nt, 200 nt or 500 nt, as the probe; and
h) nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

In a preferred embodiment, the increase of the activities of the hemoprotein according to the invention takes place by expression, preferably overexpression, in comparison with the wild type as described above, of at least one nucleic acid molecule comprising at least one nucleic acid molecule selected from the group consisting of:

a) nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) nucleic acid molecule which is obtained by amplifying a nucleic acid molecule from a cDNA database or from a genome database by means of the primers as shown in sequence No. 41 and 42;
f) nucleic acid molecule which codes for a polypeptide with hemoprotein activity and which hybridizes under stringent conditions with a nucleic acid molecule as shown in (a) to (c);
g) nucleic acid molecule coding for a hemoprotein which can be isolated from a DNA library under stringent hybridization conditions by using a nucleic acid molecule as shown in (a) to (c) or the subfragments thereof of at least 15 nt, preferably 20 nt, 30 nt, 50 nt, 100 nt, 200 nt or 500 nt, as the probe; and
h) nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

“Nucleic acids” means biopolymers of nucleotides which are linked with one another via phosphodiester bonds (polynucleotides, polynucleic acids). Depending on the type of sugar in the nucleotides (ribose or deoxyribose), a distinction is made between the two classes of the ribonucleic acids (RNA) and the deoxyribonucleic acids (DNA).

The terms “protein” and “polypeptide” are synonymous and mutually exchangeable within the meaning of the present invention.

In a preferred embodiment of the present invention, transformed cells, preferably plants, with an increased ATP/ADP ratio are produced by expressing a nonsymbiotic hemoglobin.

Nonsymbiotic hemoglobin belongs to the family of hemoglobin proteins whose function is to reversibly bind, and supply, oxygen. In contrast to leghemoglobin, it does not occur in the nodules of legumes (Leguminosae). They are involved, inter alia, in the detoxification of nitrite oxide and in the recognition of oxygen availability.

In a further preferred embodiment of the present invention, transformed cells, preferably plants, with an increased ATP/ADP ratio are produced by expressing a leghemoglobin.

Leghemoglobin belongs to the family of the hemoglobin proteins whose function is to reversibly bind, and supply, oxygen. It is derived from nodules of legumes (Leguminosae) and is a red substance which can be isolated and which resembles the myoglobin of vertebrates. By reversibly binding O₂, leghemoglobin can meet the high oxygen requirements when nitrogen is fixed by the nodule bacteria. The apoprotein is formed by the plant cells, and the heme by the bacteria (source: CD Römpp Chemie Lexikon—Version 1.0 Stuttgart/New York; Georg Thieme Verlag 1995).

In the present application, expression is taken to mean the transfer of a genetic piece of information starting from DNA or RNA into a gene product (polypeptide or protein, in the present case leghemoglobin) and is also intended to comprise the term overexpression, which means an enhanced expression so that the foreign protein or the naturally occurring protein is produced in an enhanced fashion or accounts for the majority of the total protein content of the host cell.

The expression of the hemoproteins according to the invention is achieved by the transformation of cells.

“Transformation” describes a process for introducing heterologous DNA into a prokaryotic or eukaryotic cell. A “transformed cell” describes not only the product of the transformation process, but also all transgenic progeny of the transgenic organism produced by the transformation. Thus, transformation is taken to mean the transfer of a piece of genetic information into an organism, in particular a plant. This is intended to include all the possibilities of introducing the information which are known to the skilled worker, for example microinjection, electroporation, the gene gun (particle bombardment), agrobacteria or chemical-mediated uptake (for example polyethylene-glycol-mediated DNA uptake, or via the silicon carbonate fiber technique). The genetic information may be introduced into the cells for example in the form of DNA, RNA, plasmid and other forms, and can be present either in host-genome-incorporated form as the result of recombination, in free form or independently as plasmid.

The transformation can be carried out by means of vectors comprising the abovementioned nucleic acid molecules, preferably vectors comprising expression cassettes which comprise the abovementioned nucleic acid molecules. An expression cassette comprises a nucleic acid sequence according to the invention in operable linkage with at least one genetic control element such as a promoter, and advantageously together with a further control element such as a terminator. The nucleic acid sequence of the expression cassette may be, for example, a genomic or a complementary DNA sequence or an RNA sequence, or semisynthetic or fully synthetic analogs thereof. These sequences may be present in linear or circular form, extrachromosomally or integrated into the genome. The corresponding nucleic acid sequences can be prepared synthetically or obtained naturally or comprise a mixture of synthetic and natural DNA components, and may consist of different heterologous gene segments from different organisms.

The term genetic control sequences is to be understood in the broad sense and means all those sequences which have an effect on bringing about the expression cassette according to the invention, or on the function of the latter. Genetic control sequences modify for example transcription and translation in prokaryotic or eukaryotic organisms. The expression cassettes according to the invention preferably comprise 5′- or upstream of the respective nucleic acid sequence to be expressed transgenically a promoter with one of the above-described specificities, and 3′- or downstream, a terminator sequence as additional genetic control sequence, and, if appropriate, further customary regulatory elements, in each case operably linked with the nucleic acid sequence to be expressed transgenically.

One embodiment of the present invention employs homologs of the nucleic acid molecules according to the invention.

“Homology” between two nucleic acid sequences or polypeptide sequences is identified via the identity of the nucleic acid sequence/polypeptide sequence over in each case the entire sequence length, which is calculated by comparison with the aid of the BESTFIT alignment (by the method or Needleman and Wunsch 1970, J. Mol. Biol. 48; 443-453), setting the following parameters for amino acids:

| | |
|---|---|
| Gap Weight: 50 | Length Weight: 3 |
| Average Match: 10.000 | Average Mismatch: −9.000 |

and the following parameters for nucleic acids

| | |
|---|---|
| Gap Weight: 50 | Length Weight: 3 |
| Average Match: 10.000 | Average Mismatch: 0.000 |

Instead of the term “homologous” or “homology”, the term “identity” is also used hereinbelow by way of synonym.

One embodiment of the present invention employs functional equivalents of the SEQ ID NO: 1, 3, 5. Functional equivalents according to the invention of SEQ ID NO 1, 3, 5, 7, 9, 11, 13, 15, 17, 19, 21, 23, 24, 25, 26, 27, 28, 29, 30 and/or 31 are derived by backtranslating an amino acid sequence with at least 40%, 50%, 60%, 61%, 62%, 63%, 64%, 65% or 66%, preferably at least 67%, 68%, 69%, 70%, 71%, 72% or 73%, preferably at least 74%, 75%, 76%, 77%, 78%, 79% or 80%, by preference at least 81%, 82%, 83%, 84%, 85%, 86%, 87%, 88%, 89%, 90%, 91%, 92% or 93%, especially preferably at least 94%, 95%, 96%, 97%, 98% or 99% identity with SEQ ID NO 2, 4, 6, 8, 10, 12, 14, 16, 18, 20, 22, 32, 33, 34, 35, 36, 37, 38, 39 and/or 40. Functional equivalents of SEQ ID NO 1, 3, 5, 7, 9, 11, 13, 15, 17, 19, 21, 23, 24, 25, 26, 27, 28, 29, 30 and/or 31 are encoded by an amino acid sequence which has at least 40%, 50%, 60%, 61%, 62%, 63%, 64%, 65% or 66%, preferably at least 67%, 68%, 69%, 70%, 71%, 72% or 73%, preferably at least 74%, 75%, 76%, 77%, 78%, 79% or 80%, by preference at least 81%, 82%, 83%, 84%, 85%, 86%, 87%, 88%, 89%, 90%, 91%, 92% or 93%, especially preferably at least 94%, 95%, 96%, 97%, 98% or 99% identity with the SEQ ID NO 2, 4, 6, 8, 10, 12, 14, 16, 18, 20, 22, 32, 33, 34, 35, 36, 37, 38, 39 and/or 40.

In the present context, “functional equivalents” describe nucleic acid sequences which hybridize under standard conditions with a nucleic acid sequence or parts of a nucleic acid sequence and which are capable of bringing about the expression of the hemoproteins in a cell or an organism.

To carry out the hybridization, it is advantageous to employ short oligonucleotides with a length of approximately 10-50 bp, preferably 15-40 bp, for example of the conserved or other regions, which can be determined via comparisons with other related genes in a manner known to the skilled worker. However, it is also possible to use longer fragments of the nucleic acids according to the invention with a length of 100-500 bp, or the complete sequences, for the hybridization. Depending on the nucleic acid/oligonucleotide used, the length of the fragment or the complete sequence, or depending on which type of nucleic acid, i.e. DNA or RNA, is used for the hybridization, these standard conditions vary. Thus, the melt temperatures for DNA:DNA hybrids are approximately 10° C. lower than those of DNA:RNA hybrids of the same length.

Depending on, for example, the nucleic acid, standard hybridization conditions are understood as meaning temperatures between 42 and 58° C. in an aqueous buffer solution with a concentration of between 0.1 to 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids are advantageously 0.1×SSC and temperatures of between approximately 20° C. to 65° C., preferably between approximately 30° C. to 45° C. In the case of DNA:RNA hybrids, the hybridization conditions are advantageously 0.1×SSC and temperatures of between approximately 30° C. to 65° C., preferably between approximately 45° C. to 55° C. These temperatures given for the hybridization are melting points calculated by way of example for a nucleic acid with a length of approx. 100 nucleotides and a G+C content of 50% in the absence of formamide. The experimental conditions for DNA hybridization are described in specialist genetics textbooks such as, for example, Sambrook et al., “Molecular Cloning”, Cold Spring Harbor Laboratory, 1989 and can be calculated by using formulae known to the skilled worker, for example as a function of the length of the nucleic acids, the type of the hybrids, or the G+C content. Further information regarding hybridization can be found by the skilled worker in the following textbooks: Ausubel at al. (eds.), 1985, “Current Protocols in Molecular Biology”, John Wiley & Sons, New York; Hames and Higgins (eds), 1985, “Nucleic Acids Hybridization: A Practical Approach”, IRL Press at Oxford University Press, Oxford; Brown (ed), 1991, Essential Molecular Biology: A Practical Approach, IRL Press at Oxford University Press, Oxford.

A functional equivalent is furthermore also understood as meaning nucleic acid sequences which are homologous, or identical, to a certain nucleic acid sequence (“original nucleic acid sequence”) up to a defined percentage and which have the same activity as the original nucleic acid sequences, furthermore in particular also natural or artificial mutations of these nucleic acid sequences. Relevant definitions are found at suitable places of the description.

“Mutations” of nucleic acid sequences or amino acid sequences comprise substitutions, additions, deletions, inversions or insertions of one or more nucleotide residues, as the result of which it is also possible for the corresponding amino acid sequence of the target protein to be modified by means of substitution, insertion or deletion of one or more amino acids, but where the totality of the functional properties of the target protein are essentially retained.

The term of functional equivalent comprises, in accordance with the present invention, furthermore also those nucleotide sequences which are obtained by modifying the nucleic acid sequences SEQ ID NO 1, 3, 5, 7, 9, 11, 13, 15, 17, 19, 21, 23, 24, 25, 26, 27, 28, 29, 30 and/or 31. For example, such modifications can be generated by techniques known to the skilled worker, such as site-directed mutagenesis, error-prone PCR, DNA shuffling (Nature 370, 1994, pp. 389-391) or staggered extension process (Nature Biotechnol. 16, 1989, pp. 258-261). The aim of such a modification may be for example the insertion of further restriction enzyme cleavage sites, the removal of DNA in order to truncate the sequence, the exchange of nucleotides for the purposes of codon optimation, or the addition of further sequences. Proteins which are encoded by modified nucleic acid sequences must still retain the desired functions, despite their different nucleic acid sequence.

As a consequence, functional equivalents comprise naturally occurring variants of the sequences described herein, but also artificial nucleic acid sequences, for example chemically synthesized, codon-usage-adapted nucleic acid sequences, and the amino acid sequences derived from them.

Nucleotide sequence is understood as meaning all nucleotide sequences which (i) correspond exactly to the sequences shown; or (ii) comprise at least one nucleotide sequence which corresponds to the sequences shown, within the range of the degeneracy of the genetic code; or (iii) comprise at least one nucleotide sequence which hybridizes with a nucleotide sequence which is complementary to the nucleotide sequence (i) or (ii), and, if appropriate, (iii) comprise function-neutral sense mutations in (i). In this context, the term “function-neutral sense mutations” means the exchange of chemically similar amino acids, such as, for example, glycine by alanine, or serine by threonine.

In accordance with the invention, modified forms are understood as meaning proteins in which alterations in the sequence, for example at the N and/or C terminus of the polypeptide or in the region of conserved amino acids are present, without, however, adversely affecting the function of the protein. These modifications can be carried out in the form of amino acid exchanges, using known methods.

Also included in accordance with the invention are the sequence regions which precede (5′, or upstream) and/or follow (3′, or downstream) the coding regions (structural genes). These include, in particular, sequence regions with a regulatory function. They are capable of affecting transcription, RNA stability or RNA processing, and also translation. Examples of regulatory sequences are promoters, enhancers, operators, terminators or translation enhancers, inter alia.

The present invention furthermore relates to a nucleic acid molecule which codes for a polypeptide which comprises a polypeptide which is encoded by a nucleic acid molecule comprising a nucleic acid molecule selected from the group consisting of:

a) nucleic acid molecule which codes for a polypeptide comprising the sequence shown in SEQ ID NO 6, 8, 10, 12, 14, 16, 18, 20, 22, 32, 33, 34, 35, 36, 37, 38, 39 and/or 40;
b) nucleic acid molecule which comprises at least one polynucleotide of the sequence shown in SEQ ID NO 5, 7, 9, 11, 13, 15, 17, 19, 21, 23, 24, 25, 26, 27, 28, 29, 30 and/or 31;
c) nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) nucleic acid molecule which is obtained by amplifying a nucleic acid molecule from a cDNA database or from a genome database by means of the primers as shown in sequence No. 41 and 42;
f) nucleic acid molecule which codes for a polypeptide with hemoprotein activity and which hybridizes under stringent conditions with a nucleic acid molecule as shown in (a) to (c);
g) nucleic acid molecule coding for a hemoprotein which can be isolated from a DNA library under stringent hybridization conditions by using a nucleic acid molecule as shown in (a) to (c) or the subfragments thereof of at least 15 nt, preferably 20 nt, 30 nt, 50 nt, 100 nt, 200 nt or 500 nt, as the probe; and
h) nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

The present invention furthermore relates to a polypeptide which is encoded by a nucleic acid molecule comprising a nucleic acid molecule selected from the group consisting of

a) nucleic acid molecule which codes for a polypeptide comprising the sequence shown in SEQ ID NO 6, 8, 10, 12, 14, 16, 18, 20, 22, 32, 33, 34, 35, 36, 37, 38, 39 and/or 40;
b) nucleic acid molecule which comprises at least one polynucleotide of the sequence shown in SEQ ID NO 5, 7, 9, 11, 13, 15, 17, 19, 21, 23, 24, 25, 26, 27, 28, 29, 30 and/or 31;
c) nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) nucleic acid molecule which is obtained by amplifying a nucleic acid molecule from a cDNA database or from a genome database by means of the primers as shown in sequence No. 41 and 42;
f) nucleic acid molecule which codes for a polypeptide with hemoprotein activity and which hybridizes under stringent conditions with a nucleic acid molecule as shown in (a) to (c);
g) nucleic acid molecule coding for a hemoprotein which can be isolated from a DNA library under stringent hybridization conditions by using a nucleic acid molecule as shown in (a) to (c) or the subfragments thereof of at least 15 nt, preferably 20 nt, 30 nt, 50 nt, 100 nt, 200 nt or 500 nt, as the probe; and
h) nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

The present invention furthermore relates to a nucleic acid molecule which codes for a polypeptide which comprises a polypeptide which is encoded by a nucleic acid molecule

which differs in one, two, three, four, five, six, seven, eight, nine, ten or more nucleic acids from a nucleic acid molecule comprising a nucleic acid molecule selected from the group consisting of
a) nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) nucleic acid molecule which comprises at least one polynucleotide of the sequence shown in SEQ ID NO 1, 3, 5, 7, 9, 11, 13, 15, 17, 19, 21, 23, 24, 25, 28, 27, 28, 29, 30 and/or 31;
c) nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) nucleic acid molecule which is obtained by amplifying a nucleic acid molecule from a cDNA database or from a genome database by means of the primers as shown in sequence No. 41 and 42;
f) nucleic acid molecule which codes for a polypeptide with hemoprotein activity and which hybridizes under stringent conditions with a nucleic acid molecule as shown in (a) to (c);
g) nucleic acid molecule coding for a hemoprotein which can be isolated from a DNA library under stringent hybridization conditions by using a nucleic acid molecule as shown in (a) to (c) or the subfragments thereof of at least 15 nt, preferably 20 nt, 30 nt, 50 nt, 100 nt, 200 nt or 500 nt, as the probe; and
h) nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45;
and which codes for a polypeptide with the activity of a hemoprotein.

The present invention furthermore relates to a polypeptide with the activity of a hemoprotein which is encoded by a nucleic acid molecule

which differs in one, two, three, four, five, six, seven, eight, nine, ten or more nucleic acids from a nucleic acid molecule comprising a nucleic acid molecule selected from the group consisting of
a) nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) nucleic acid molecule which is obtained by amplifying a nucleic acid molecule from a cDNA database or from a genome database by means of the primers as shown in sequence No. 41 and 42;
f) nucleic acid molecule which codes for a polypeptide with hemoprotein activity and which hybridizes under stringent conditions with a nucleic acid molecule as shown in (a) to (c);
g) nucleic acid molecule coding for a hemoprotein which can be isolated from a DNA library under stringent hybridization conditions by using a nucleic acid molecule as shown in (a) to (c) or the subfragments thereof of at least 15 nt, preferably 20 nt, 30 nt, 50 nt, 100 nt, 200 nt or 500 nt, as the probe; and
h) nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45; and which codes for a polypeptide with the activity of a hemoprotein.

The present invention furthermore relates to a DNA expression cassette comprising a nucleic acid sequence as described above.

The present invention furthermore relates to a vector comprising an expression cassette comprising a nucleic acid sequence as described above.

The present invention also relates to a cell within the meaning of the invention, preferably a monocotyledonous organism or a dicotyledonous organism, with an increased activity of at least one hemoprotein based on the expression of a nucleic acid sequence as described above.

The present invention furthermore relates to a cell generated by the method according to the invention.

In one embodiment of the present invention, the alteration of the activity of a hemoprotein brings about not only an increased ATP/ADP ratio, but also an increase in the oil content in the cells.

The oil content relates to the total fatty acid content in the cells according to the invention.

Within the meaning of the invention, “oil” comprises neutral and/or polar lipids and mixtures of these. Those listed in table 1 may be mentioned by way of example, but not by limitation.

TABLE 1
Classes of plant lipids

| | |
|---|---|
| Neutral lipids | Triacylglycerol (TAG) |
| | Diacylglycerol (DAG) |
| | Monoacylglycerol (MAG) |
| Polar lipids | Monogalactosyldiacylglycerol (MGDG) |
| | Digalactosyldiacylglycerol (DGDG) |
| | Phosphatidylglycerol (PG) |
| | Phosphatidylcholine (PC) |
| | Phosphatidylethanolamine (PE) |
| | Phosphatidylinositol (PI) |
| | Phosphatidylserine (PS) |
| | Sulfoquinovosyldiacylglycerol |

Neutral lipids preferably refers to triacylglycerides. Both neutral and polar lipids may comprise a wide range of various fatty acids. The fatty acids listed in table 2 may be mentioned by way of example, but not by limitation.

TABLE 2
Overview over various fatty acids (selection)

| Nomenclature¹ | Name |
|---|---|
| 16:0 | Palmitic acid |
| 16:1 | Palmitoleic acid |
| 16:3 | Roughanic acid |
| 18:0 | Stearic acid |
| 18:1 | Oleic acid |
| 18:2 | Linoleic acid |
| 18:3 | Linolenic acid |
| γ-18:3 | Gamma-linolenic acid* |
| 20:0 | Arachidic acid |
| 22:6 | Docosahexanoic acid (DHA)* |
| 20:2 | Eicosadienoic acid |
| 20:4 | Arachidonic acid (AA)* |
| 20:5 | Eicosapentaenoic acid (EPA)* |
| 22:1 | Erucic acid |

¹Chain length: number of double bonds
*not naturally occurring in plants

As regards more detailed information, reference is also made to Römpp Chemie Lexikon—CD Version 2.0, Stuttgart/New York: Georg Thieme Verlag 1999.

In a preferred variant, the unsaturated fatty acid content, in particular the linolenic acid content, is increased.

However, the total protein content is not reduced, or to a small extent only, by increasing the total oil content of the cell according to the invention. This means that the total fatty acid content expressed in weight by weight dry weight, is significantly increased over that of the wild type. However, the total protein content in comparison with that of the wild type, also expressed as weight by weight dry weight, remains constant or is reduced to a negligible extent only. Based on the wild type, the reduction, as a percentage, is less than the increase of the oil content.

The increase of the ATP/ADP ratio, that is to say the increase of the energy status as the result of the storage of energy in ATP, remains constant in cells which, owing to the method according to the invention, show increased activity of hemoproteins. This means that the ATP/ADP ratio of the cells is not affected by a modification of the external conditions.

External conditions are to be understood as meaning, for the purposes of the invention, the culture conditions for cells, tissues, organs, microorganisms or plants. They may take the form of, for example, media composition, temperature, composition of the atmosphere, or other factors which affect the wild type.

In one embodiment of the present invention, the ATP/ADP ratio of the cells with an increased hemoprotein activity according to the invention is, when the oxygen concentration in the surrounding atmosphere is reduced to 4%, at least 200%, 300%, preferably 400%, especially preferably at least 500% or more, based on the ATP/ADP ratio of the wild type.

In addition, the amount of lactate formed under these anaerobic culture conditions is no more than 80%, preferably 75%, 70%, especially preferably 65%, 60%, 55%, 50% or less, based on the amount of lactate of the wild type.

In a further embodiment of the invention, the modification of the hemoprotein activity, the increased ATP/ADP ratio, the increased oil content and/or the reduced lactate quantity are a stable feature of the cells according to the invention which is retained over several generations, preferably up to the T2, especially up to the T3 generation.

In a preferred variant of the present invention, the cells according to the invention are plant cells, organs, plant parts or intact plants.

Within the scope of the invention, “plants” means all dicotyledonous or monocotyledonous plants. “Plants” within the meaning of the invention are plant cells, plant tissue, plant organs or intact plants, such as seeds, tubers, flowers, pollen, fruits, seedlings, roots, leaves, stems or other plant parts. Plants is furthermore taken to mean propagation material such as seeds, fruits, seedlings, cuttings, tubers, cuttings or rootstocks.

Also embraced by the term “plants” are the mature plants, seeds, shoots and seedlings, and also their derived parts, propagation material, plant organs, tissue, protoplasts, callus and other cultures, for example cell cultures, and all other types of groups of plant cells which give functional or structural units. Mature plants means plants at any developmental stage beyond that of the seedlings. Seedling means a young, immature plant at an early developmental stage.

“Plant” also comprises annual and perennial dicotyledonous or monocotyledonous plants and includes by way of example, but not by limitation, those of the genera Bromus, Asparagus, Pennisetum, Lolium, Oryza, Zea, Avena, Hordeum, Secale, Triticum, Sorghum and Saccharum.

In a preferred embodiment, the method is applied to monocotyledonous plants, for example from the family, Poaceae, especially preferably to the genera Oryza, Zea, Avena, Hordeum, Secale, Triticum, Sorghum and Saccharum, very especially preferably to plants of agricultural importance such as, for example, Hordeum vulgare (barley), Triticum aestivum (wheat), Triticum aestivum subsp.spelta (spelt), Triticale, Avena sative (oats), Secale cereale (rye), Sorghum bicolor (sorghum), Zea mays (maize), Saccharum officinarum (sugarcane) or Oryza sativa (rice). Preferred monocotyledonous plants are especially selected among the monocotyledonous crop plants, such as, for example, the family Gramineae, such as rice, maize, wheat or other cereal species such as barley, sorghum/millet, rye, triticale or oats, and sugarcane, and all types of grasses. Especially preferred from the family Gramineae are rice, maize, wheat and barley.

Thus, a transformed plant according to the invention is a genetically modified plant.

In accordance with the invention, all plants are suitable for carrying out the method according to the invention. The following are preferably used: potatoes, Arabidopsis thaliana, oilseed rape, soybeans, peanuts, maize, cassaya, physic nut, yams, rice, sunflowers, rye, barley, hops, oats, durum wheat and aestivum wheat, lupins, peas, clover, beet, cabbage, grapevines and the like, as they are known for example from the ordinance on the species list of the Saatgutverkehrsgesetz [Seed Trade Act] (Blatt für PMZ [Journal of Patent, Models and Trademark Affairs] 1986 p. 3, last updated Blatt liar PMZ 2002 p. 68).
1. Preferred dicotyledonous plants are selected in particular from the dicotyledonous crop plants such as, for example,
Asteraceae, such as sunflowers, tagetes or calendula,
Compositae, especially the genus Lactuca, very particularly the species sativa (lettuce),
Cruciferae, especially the genus Brassica, very especially the species napus (oilseed rape), campestris (beet), oleracea cv Tastie (cabbage), oleracea cv Snowball Y (cauliflower) and oleracea cv Emperor (broccoli) and other cabbages; and of the genus Arabidopsis, very especially the species thaliana, and cress or canola,
Cucurbitaceae such as melon, pumpkin/squash or zucchini,
Leguminosae especially the genus Glycine, very especially the species Glycine max (soybean), and alfalfa, pea, beans or peanut,
Rubiaceae, preferably the subclass Lamidae, such as, for example, Coffea arabica or Coffea liberica (coffee bush),
Solanaceae, especially the genus Lycopersicon, very especially the species esculentum (tomato) and the genus Solanum, very especially the species tuberosum (potato) and melongena (aubergine), and tobacco or capsicum,
Sterculiaceae, preferably the subclass Dilleniidae, such as, for example, Theobroma cacao (cacao bush),
Theaceae, preferably the subclass Dilleniidae, such as, for example, Camellia sinensis or Thea sinensis (tea bush),
Umbelliferae, especially the genus Daucus (very especially the species carota (carrot) and Apium (very especially the species graveolens dulce (celery)) and others; and the genus Capsicum, very especially the species annuum (pepper),
and linseed, soya, cotton, hemp, flax, cucumber, spinach, carrot, sugarbeet, and the various tree, nut and grapevine species, in particular banana and kiwi.

Also encompassed are ornamental plants, useful or ornamental trees, flowers, cut flowers, shrubs or turf. The following may be mentioned by way of example but not by limitation: angiosperms, bryophytes such as, for example, Hepaticae (liverworts) and Musci (mosses); pteridophytes such as ferns, horsetail and lycopods; gymnosperms such as conifers, cycades, ginkgo and Gnetatae; the families of the Rosaceae such as rose, Ericaceae such as rhododendrons and azaleas, Euphorbiaceae such as poinsettias and Groton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geranium, Liliaceae such as dracaena, Moraceae such as ficus, Araceae such as cheeseplant and many others.

It is especially preferred to use oil crops, i.e. plants whose oil content is already naturally high and/or which can be used for the industrial production of oils. These plants can have a high oil content and/or else a particular fatty acid composition which is of interest industrially. Preferred plants are those with a lipid content of at least 1% by weight. Oil crops encompass by way of example: Borago officinalis (borage); Brassica species such as B. campestris, B. napus, B. rapa (mustard or oilseed rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (physic nut or purging nut), Linum usitatissimum (linseed or flax); Oenothera biennis (evening primrose); Olea europaea (olive); Oryza sativa (rice); Ricinus communis (castor); Sesamum indicum (sesame); Triticum species (wheat); Zea mays (maize), and various nut species such as, for example, walnut or almond.

When the plants used are plants which belong to the genus Leguminosae (legumes), then the expression of foreign proteins leghemoglobins or hemoglobins which do not occur symbiotically in nature or the modification of the plants such that they overexpress the naturally occurring leghemoglobin or nonsymbiotic hemoglobin come within the scope of the invention.

Most preferred are potatoes, Arabidopsis thaliana, oilseed rape and soya.

It is advantageous when the abovementioned plants express a leghemoglobin selected from the group consisting of leghemoglobin from the plants Lupinus luteus (LGB1_LUPLU, LGB2_LUPLU), Glycine max (LGBA_SOYBN, LGB2_SOYBN, LGB3_SOYBN), Medicago sativa (LGB1-4_MEDSA), Medicago trunculata (LGB1_MEDTR), Phaseolus vulgaris (LGB1_PHAVU, LGB2_PHAVU), Vicia faba (LGB1_VICFA, LGB2_VICFA), Pisum sativum (LGB1_PEA, LGB2_PEA), Vigna unguiculata (LGB1_VIGUN), Lotus japonicus (LGB_LOTJA), Psophocarpus tetragonolobus (LGB_PSOTE), Sesbania rostrata (LGB1—SESRO), Casuarina glauca (HBPA_CASGL) and Canvalaria lineata (HBP_CANLI). The Swiss-Prot database entries are given in parentheses.

It is especially advantageous when the abovementioned plants express a nonsymbiotic hemoglobin selected from the group consisting of hemoglobin from the plants Arabidopsis thaliana (AT_AHB2), Brassica napus (BN_AHB2), Linum usitatissimum (LU_AHB2), Glycine max (GM_AHB2), Helianthus annuus (HA_AHB2), Triticum aestivum (TA_AHB2), Hordeum vulgare (HV_AHB2), Oryza sativa (OS_AHB2) and Zea mays (ZM_AHB2).

Plants with the sequence No. 1 (AT-AHB2) coding for nonsymbiotic hemoglobin are especially advantageous.

In a preferred variant of the invention, they are plants which express the hemoprotein in a reserve-organ-specific manner.

These are, for example, bulbs, tubers, seeds, grains, nuts, leaves and the like. Storage organs within the meaning of the invention also mean fruits. Fruits are the collective name for the plant organs which surround the seed as nutritive tissue. Here, one considers not only the edible fruits, in particular dessert fruit, but also legumes, cereals, nuts, spices, but also legally used drugs (see fructus, semen). Naturally, the reserve substances can also be stored in all of the plant.

The hemoprotein is preferably expressed in a tuber-specific or seed-specific manner.

Suitable plants are all those mentioned above. It is especially preferred when they are tuber-producing plants, in particular potato plants, or seed-producing plants, in particular Arabidopsis thaliana or oilseed rape.

The tissue-specific expression can be achieved for example by using a tissue-specific promoter. Such a tissue-specific expression is known for example from U.S. Pat. No. 6,372,961 B1 column 11, lines 44 at seq.

in a further embodiment, the present invention relates to the use of the above-described nucleic acid molecules coding for polypeptides with the activity of hemoproteins for the production of cell, tissue, organ, microorganism or plant with an increased ATP/ADP ratio and/or modified oil content, preferably increased fatty acid content, preferably increased linolenic acid content.

The invention is described by way of example with reference to the following experiment.

### EXAMPLES

### General Methods

Unless, otherwise specified, all chemicals are obtained from Fluke (Buchs), Merck (Darmstadt), Roth (Karlsruhe), Serve (Heidelberg) and Sigma (Deisenhofen). Restriction enzymes, DNA-modifying enzymes and molecular biology kits were obtained from Amersham-Pharmacia (Freiburg), Biometra (Göttingen), Roche (Mannheim), New England Biolabs (Schwalbach), Novagen (Madison, Wis., USA), Perkin-Elmer (Weiterstadt), Qiagen (Hilden), Stratagen (Amsterdam, Netherlands), Invitrogen (Karlsruhe) and Ambion (Cambridgeshire, United Kingdom). The reagents used were employed following the manufacturers' instructions.

The chemical synthesis of oligonucleotides can be effected for example in the known manner using the phosphoamidite method (Voet, Voet, 2nd edition, Wiley Press New York, page 896-897). The cloning steps carried out within the scope of the present invention such as, for example, restriction cleavages, agarose gel electrophoresis, purification of DNA fragments, transfer of nucleic acids to nitrocellulose and nylon membranes, linkage of DNA fragments, transformation of E. coli cells, bacterial cultures, phage propagation and sequence analysis of recombinant DNA are carried out as described by Sambrook at al. (1989) Cold Spring Harbor Laboratory Press; ISBN 0-87969-309-6. Recombinant DNA molecules are sequenced with a laser fluorescence DNA sequencer from ABI, following the method of Sanger (Sanger et al. (1977) Proc Nail Aced Sci USA 74:5463-5467).

### Example 1

### Cloning the AHB1 and AHB2 Genes from Arabidopsis thaliana

To clone the AHB2 gene, the total RNA from 6-week old Arabidopsis plants was extracted. The corresponding cDNA was prepared by RT-PCR with the aid of SUPERSCRIPT II (Invitrogen).

To clone the AHB2 gene, the Arabidopsis cDNA which has been isolated was employed in a PCR reaction, using the oligonucleotide primers AHb2f and AHb2r.

### Sequence Primer Ahb2f

SEQ. ID. No:
5′-TTTGGTACCATGGGEGAGATTGGGTTTACAGAG-3′

### Sequence Primer Ahb2r

SEQ. ID. No:
5′-TTTGGATCCTTATGACCTTTCTTGTTTCATCTCGG-3′

Composition of the PCR Mix (50 μl):

5.00 μl cDNA from Arabidopsis thaliana
5.00 μl 10× buffer (Advantage Polymerase)+25 mM MgCl₂
5.00 μl 2 mM dNTP
1.25 μl of each primer (10 pmol/μl)

### 0.50 μl Advantage Polymerase

The polymerase employed was the Advantage Polymerase from Clontech.

PCR program:

Initial denaturation for 2 min at 95° C., then 35 cycles of 45 sec at 95° C., 45 sec at 55° C. and 2 min at 72° C. Final extension. 5 min at 72° C.

Thereafter, the PCR mixtures were separated via agarose gel electrophoresis, and the amplified DNA fragments of AHB2 were excised from the gel, purified with the “Gelpurification” kit from Qiagen following the manufacturer's instructions and eluted with 50 μl of elution buffer.

Thereafter, the DNA fragment was cloned into the vector pCR2.1-TOPO (Invitrogen) following the manufacturer's instructions, resulting in the vector pCR2.1-AHB2, and the sequence was verified by sequencing.

Thereafter, the coding sequences for AHB2 were cloned into a binary plant vector such as pBIN downstream of the seed-specific USP promoter (Baumein et al. (1991) Mol Gen Genet. 225(3):459-467). To this end, the vector pCR2.1-AHB2 was digested with the restriction enzymes KpnI and BamHI. The resulting DNA fragments were separated by agarose gel electrophoresis, and the AHB-encoding fragments were excised from the gel, purified with the “Gelpurification” kit from Qiagen following the manufacturer's instructions and eluted with 50 μl of elution buffer. The eluted DNA fragments were ligated (T4 ligase from New England Biolabs) overnight at 16° C. with the binary vector which had been digested with the same enzymes. The ligation products are then transformed into TOP10 cells (Stratagene) following the manufacturer's instructions and selected in a suitable manner. Positive clones are verified by PCR and sequencing, using the primers AHb2f and AHb2r.

### Example 3

### Transformation of Agrobacterium

The Agrobacterium-mediated transformation of plants can be effected for example using the Agrobacterium tumefaciens strains GV3101 (pMP90) (Koncz and Schell (1986) Mol Gen Genet. 204: 383-396) or LBA4404 (Clontech). The transformation can be effected by standard transformation techniques (Deblaere et al. (1984) Nucl Acids Res 13:4777-4788).

### Example 4

### Plant Transformation

The Agrobacterium-mediated transformation of Arabidopsis thaliana was carried out using standard transformation and regeneration techniques (Gelvin, Stanton B., Schilperoort, Robert A., Plant Molecular Biology Manual, 2nd Edition, Dordrecht: Kluwer Academic Publ., 1995, in Sect., Ringbuch Zentrale Signatur: BT11-P ISBN 0-7923-2731-4; Glick, Bernard R., Thompson, John E., Methods In Plant Molecular Biology and Biotechnology, Boca Raton: CRC Press, 1993, 360 p., ISBN 0-8493-5164-2). The use of antibiotics for the selection of agrobacteria and plants depends on the binary vectory and the Agrobacterium strain used for the transformation. The selection of the AHB2 transformed Arabidopsis thaliana plants was carried out with hygromycin.

The Agrobacterium-mediated transformation of oilseed rape can be effected for example by cotyledon or hypocotyl transformation (Moloney at al., Plant Cell Report 8 (1989) 238-242; De Block et al., Plant Physiol. 91 (1989) 694-701). The use of antibiotics for the selection of agrobacteria and plants depends on the binary vectory and the Agrobacterium strain used for the transformation.

The Agrobacterium-mediated transfer of genes into linseed (Linum usitatissimum) can be effected using, for example, a technique described by Mlynarova et al. (1994) Plant Cell Report 13:282-285.

The transformation of soybeans can be effected using, for example, a technique described in EP-A-0 0424 047 (Pioneer Hi-Bred International) or in EP-A-0 0397 687, U.S. Pat. No. 5,376,543, U.S. Pat. No. 5,169,770 (University Toledo).

The transformation of plants using particle bombardment, polyethylene-glycol-mediated DNA uptake or the silicon carbonate fiber technique is described, for example, by Freeling and Walbot “The maize handbook” (1993) ISBN 3-540-97826-7, Springer Verlag New York).

### Example 5

### Analysis of the Expression of a Recombinant Gene Product in a Transformed Organism

A suitable method of determining the transcription level of the gene (an indication of the amount of RNA which is available for the translation of the gene product) is to carry out a Northern blot as specified hereinbelow (for reference, see Ausubel et al. (1988) Current Protocols in Molecular Biology, Wiley: New York, or the examples section mentioned above), where a primer, which is such that it binds to the gene of interest, is labeled with a detectable marker (usually radioactive or chemiluminescent), so that, when the total RNA of a culture of the organism is extracted, separated on a gel, transferred to a stable matrix and incubated with this probe, the binding and the extent of the binding of the probe indicates the presence and also the amount of the mRNA for this gene. This information indicates the transcription level of the transformed gene. Cellular total RNA can be prepared from cells, tissue or organs by a variety of methods, all of which are known in the art, for example the method described by Bormann, E. R. et al. (1992) Mol. Microbial. 6:317-326.

### Northern Hybridization:

To carry out the RNA hybridization, total RNA was extracted from maturing seeds with the aid of the Concert RNA Plant Reagent (Invitrogen GmbH, Karlsruhe, Germany). 20 μg of total RNA or 1 μg of poly(A)+ RNA were separated by gel electrophoresis in agarose gels with a strength of 1.25% using formaldehyde, as described in Amasino (1986, Anal. Biochem. 152, 304), transferred to positively charged nylon membranes (Hybond N+, Amersham, Brunwick) by capillarity using 10×SSC, immobilized by means of UV light and prehybridized for 3 hours at 68° C. using hybridization buffer (10% dextran sulfate w/v, 1 M NaCl, 1% SDS, 100 mg herring sperm DNA). Labeling of the DNA probe using the Highprime DNA labeling kit (Roche, Mannheim, Germany) was carried out during prehybridization, using α-³²P dCTP (Amersham Pharmacia, Brunswick, Germany). After the labeled DNA probe had been added, the hybridization was carried out in the same buffer at 68° C. overnight. The wash steps were carried out twice for 15 min using 2×SSC and twice for 30 min using 1×SSC, 1% SDS, at 68° C. The exposure of the sealed filters was carried out at −70° C. for a period of 1 to 14 days.

Standard techniques, such as a Western blot, may be employed to analyze the presence or the relative amount of protein translated from this mRNA (see, for example, Ausubel at al. (1988) Current Protocols in Molecular Biology, Wiley: New York). In this method, the cellular total proteins are extracted, separated by means of gel electrophoresis, transferred to a matrix such as nitrocellulose, and incubated with a probe, such as an antibody, which binds specifically to the protein in question. Usually, this probe is provided with a chemiluminescent or colorimetric marker which can be detected readily. The presence and the amount of the marker observed indicates the presence and the amount of the desired protein which is present in the cell.

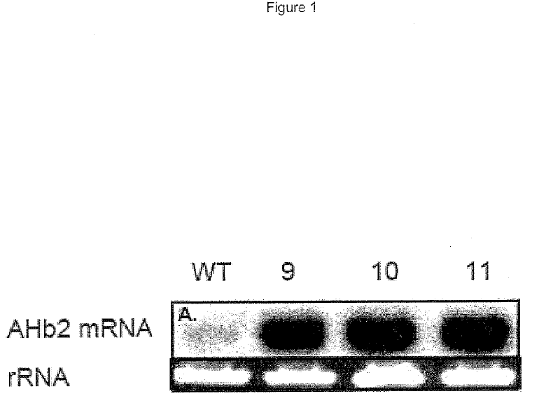

FIG. 1 shows the results of the Northern blot of 3 independent transgenic Arabidopsis lines which have been transformed with the AHB2 construct, and of the wild type. The plants of lines 9, 10 and 11 revealed a strong detection signal in the Northern blot. Accordingly, the plants express the AHB2 gene in maturing seeds. In the seed sample of the wild type, in contrast, only a weak signal was detected; which was based on the expression of the endogenous AHB2 gene.

### Example 6

### Analysis of the Effect of the Recombinant Proteins on the Production of the Desired Product

The effect of the genetic modification in plants, or on the production of a desired compound (such as a fatty acid), can be determined by growing the modified plant under suitable conditions (like the conditions described above) and by examining the medium and/or the cellular components for the increased production of the desired products (i.e. of lipids or a fatty acid). These analytical techniques are known to the skilled worker and comprise spectroscopy, thin-layer chromatography, various types of staining methods, enzymatic and microbiological methods, and analytic chromatography such as high-performance liquid chromatography (see, for example, Ullman, Encyclopedia of Industrial Chemistry, Vol. A2, p. 89-90 and p. 443-613, VCH: Weinheim (1985); Fallon, A., et at., (1987) “Applications of HPLC in Biochemistry” in: Laboratory Techniques in Biochemistry and Molecular Biology, Vol. 17; Rehm at al. (1993) Biotechnology, Vol. 3, Chapter “Product recovery and purification”, p. 469-714, VCH: Weinheim; Baiter, P. A., et al. (1988) Bioseparations: downstream processing for Biotechnology, John Wiley and Sons; Kennedy, J. F., und Cabral, J. M. S. (1992) Recovery processes for biological Materials, John Wiley and Sons; Shaeiwitz, J. A. und Henry, J. D. (1988) Biochemical Separations, in: Ullmann's Encyclopedia of Industrial Chemistry, Vain; Chapter 11, p. 1-27, VCH: Weinheim; and Dechow, F. J. (1989) Separation and purification techniques in biotechnology, Noyes Publications).

Besides the abovementioned methods, plant lipids are extracted from plant material as described by Cahoon et al. (1999) Proc. Natl. Acad. Sol. USA 96 (22):12935-12940 and Browse et al. (1986) Analytic Biochemistry 152:141-145. The qualitative and quantitative lipid or fatty acid analysis is described by Christie, William W., Advances in Lipid Methodology, Ayr/Scotland: Oily Press (Oily Press Lipid Library; 2); Christie, William W., Gas Chromatography and Lipids. A Practical Guide-Ayr, Scotland: Oily Press, 1989, Repr. 1992, IX, 307 p. (Oily Press Lipid Library; 1); “Progress in Lipid Research, Oxford: Pergamon Press, 1 (1952)-16 (1977) under the title: Progress in the Chemistry of Fats and Other Lipids OMEN.

An example is the analysis of fatty acids (abbreviations: FAME, fatty acid methyl ester; GC-MS: gas liquid chromatography/mass spectrometry; TAG, triacylglycerol; TLC, thin-layer chromatography).

Unambiguous proof of the presence of fatty acid products can be obtained by analyzing recombinant organisms by analytical standard methods: GC, GC-MS or TLC, as described on several occasions by Christie and the references cited therein (1997, in: Advances on Lipid Methodology, fourth edition: Christie, Oily Press, Dundee, 119-169; 1998, Gaschromatographie-Massenspektrometrie-Verfahren [Gas chromatography/mass spectrometry methods], Lipide 33:343-353).

The material to be analyzed can be disrupted by sonication, milling in the glass mill, liquid nitrogen and milling or other applicable methods. After disruption, the material must be centrifuged. The sediment is resuspended in distilled water, heated for 10 minutes at 100° C., cooled on Ice and recentrifuged, followed by extraction In 0.5 M sulfuric acid in methanol with 2% dimethoxypropane for 1 hour at 90° C., which gives hydrolyzed oil and lipid compounds, which give transmethylated lipids. These fatty acid methyl esters are extracted in petroleum ether and finally subjected to GC analysis using a capillary column (Chrompack, WCOT Fused Silica, CP-Wax-52 CB, 25 mikrom, 0.32 mm) at a temperature gradient of between 170° C. and 240° C. for 20 min and for 5 min at 240° C. The identity of the fatty acid methyl esters obtained must be defined using standards which are available from commercial sources (i.e. Sigma).

Plant material is first homogenized mechanically with a pestle and mortar to make it more accessible to extraction.

The following protocol was used for the quantitative and qualitative oil analysis of the Arabidopsis plants transformed with the constructs ADH1 and ADH2:

Lipid extraction from the seeds is carried out by the method of Bligh & Dyer (1959) Can Biochem Physiol 37:911. To this end, 5 mg of Arabidopsis seeds are weighed into 1.2 ml Qiagen microtubes (Qiagen, Hilden) using a Sartorius (Göttingen) microbalance. The seed material is homogenized with 1 ml chloroform/methanol (1:1; contains mono-C15-glycerol from Sigma as internal standard) in an MM300 Retsch mill from Retsch (Haan) and incubated for 20 min at RT. After centrifugation, the supernatant was transferred into a fresh vessel, and the sediment was reextracted with 1 ml of chloroform/methanol (1:1). The supernatants were combined and evaporated to dryness. The fatty acids were derivatized by means of acidic methanolysis. To this end, the extracted lipids were treated with 0.5 M sulfuric acid in methanol and 2% (v/v) dimethoxypropane and incubated for 60 min at 80° C. This was followed by two extractions with petroleum ether, followed by wash steps with 100 mM sodium hydrogen carbonate and water. The fatty acid methyl esters thus prepared were evaporated to dryness and taken up in a defined volume of petroleum ether. Finally, 2 μl of the fatty acid methyl ester solution were separated by gas chromatography (FIR 6890, Agilent Technologies) on a capillary column (Chrompack, WCOT Fused Silica, CP-Wax-52 CB, 25 m, 0.32 mm) and analyzed by a flame ionization detector.

The oil was quantified by comparing the signal strengths of the derivatized fatty acids with those of the internal standard Mono-C15-glycerol (Sigma).

The fatty acid profile was determined by comparing the signal strengths relatively to one another. The determination of the unsaturation/saturation index (USI) was carried out as described by Gutlérrez at al. ((2005) Food Chemistry 90, 341-346) and reflects the ratio of unsaturated to saturated fatty acids in the seed oil.

The quantitative protein analysis of the Arabidopsis plants transferred with the construct USP-AHB2 was carried out using the protocol of Bradford (1976). The standard used was bovine serum albumin,

TABLE 3
Oil content (total fatty acid content) in matured and maturing
(13-14 DAF) seeds of transgenic Arabidopsis lines which
have been transformed with the construct USP-AHB2 and in
mature and maturing (13-14 DAF) seeds of untransformed
wild type plants. The oil content in mature seeds was determined
over three successive generations. The data shown are means
and standard deviations from 6 independent measurements.
Significant differences to the wild type (based on the statistic t-test
analysis; p < 0.05) are identified by an asterisk (*).

| Lipid content | | | | |
|---|---|---|---|---|
| (mg TFA gDW⁻¹) | WT | Line 9 | Line 10 | Line 11 |
| Lipid content in | | | | |
| mature seed | | | | |
| T1 generation | 324 ± 12 | 430 ± 20* | 488 ± 79* | 502 ± 34* |
| T2 generation | 383 ± 13 | 451 ± 48 | 499 ± 21* | 507 ± 30* |
| T3 generation | 331 ± 26 | 380 ± 18 | 435 ± 17* | 464 ± 25* |
| Lipid content in | | | | |
| developing seed | | | | |
| T3 generation | 128 ± 11 | 245 ± 22* | 224 ± 13* | 208 ± 34* |

Table 3 compiles by way of example the course of the oil contents in mature seeds of 3 independent transgenic Arabidopsis lines over 3 generations which had been transformed with the construct USP-AHB2, and of the untransformed wild-type plants. The data are the means of 6 independent measurements. The standard deviations are also shown. Significant differences to the wild type (based on the statistic t-test analysis) are identified by asterisks (*). In all 3 generations, a pronounced increase in the oil content was demonstrated in the mature seeds of the transgenic lines. Accordingly, the phenotype obtained is stable over several generations. In addition, a markedly higher oil content in the transgenic lines was also found in maturing T3 seeds during the oil storage phase (see table 1).

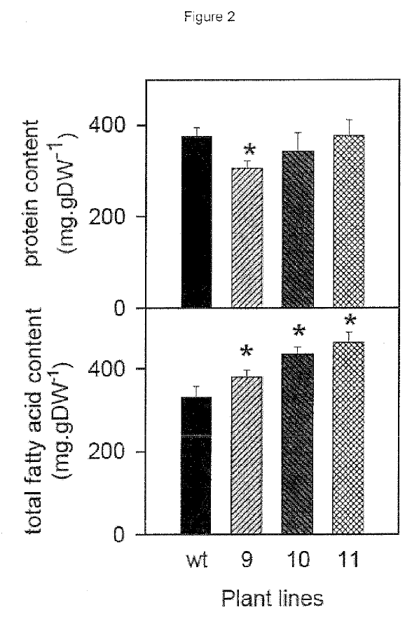

FIG. 2 shows by way of example the results for the quantitative determination of the oil and protein contents in T3 seeds of 3 independent transgenic Arabidopsis lines (9, 10, 11) which had been transformed with the construct USP-AHB2, and in the seeds of the untransformed wild-type plants. The data are the means of 10 independent measurements. The standard deviations are also shown. Significant differences to the wild type (based on the statistic t-test analysis) are identified by asterisks (*). A significant increase in the oil content by 15% (line 9), 31% (line 10) and 40% (line 11) was found in all three transgenic lines. The different increases in the oil content of the various lines correlate with the expression levels shown in FIG. 2. In contrast, the overexpression of AHB2 has no effect on the oil content.

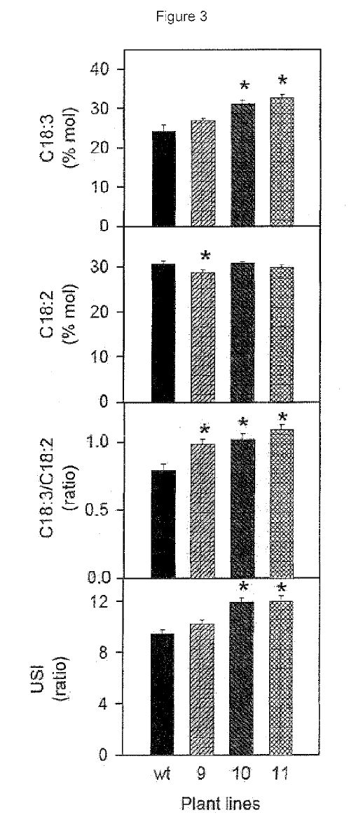

FIG. 3 shows by way of example the results of the qualitative oil analysis in the mature seeds of transgenic Arabidopsis lines which have been transformed with the construct USP-AHB2, and in the seeds of the untransformed wild-type plants (A. linoleic acid content, B. linolenic acid content, C. linoleic/linolenic acid ratio, and D. USI (unsaturation/saturation index)). The data are the means and standard deviations of 10 independent measurements. Significant differences to the wild type (based on the statistic t-test analysis) are identified by asterisks (*). The seed-specific overexpression of AHB2 leads to a marked increase of a-linolenic acid (C18:3) in the seed oil from 25% in the wild-type plant to over 30% in the transgenic lines 10 and 11. In contrast, the linolenic acid content (C18:2), the precursor of C18:3, is unchanged. This is also reflected in the C18:3/C18:2 ratio (0.8 in the seed oil of the wild-type plants, and >1 in the seed oil of the transgenic plants). Accordingly, the overexpression of AHB2 leads to an increased desaturation of the fatty acids in the seeds of the transgenic lines, as also reflected by the USI, which climbs from 9 in the wild-type seeds to up to 12 in the transgenic seeds.

### Example 7

### Determination of the ATP/ADP Ratio and of the Lactate Content

To study the effect of different oxygen concentrations on the metabolite in the seeds of the wild type and of AHB2-overexpressing Arabidopsis plants, the plants were grown in the greenhouse (21° C./day and 17° C./night, 50% humidity day and night, photoperiod 16 h day/8 h night, night intensity 180 μmol photons m⁻²s⁻¹. To carry out the incubation experiments with different oxygen concentrations, pod-bearing stems were placed into a transparent plastic bag in which air with an oxygen content of 21% or 4% (v/v) was circulating. The air mixtures from Messer Griesheim GmbH (Magdeburg, Germany) contained 350 ppm CO₂, oxygen concentrations as stated above and nitrogen. After 2 hours, the pods were harvested and immediately shock-frozen in liquid nitrogen. Seeds were dissected from 13-14-day-old lyophilized pods as described by Gibon et al. (2002) Plant J 30:221-235.

To analyze the metabolites ATP, ADP and lactate, seeds were homogenized in a mixer mill, cooled with liquid nitrogen, from Retsch (Haan, Germany) and subsequently extracted with trichloroacetic acid. The quantification of the metabolites was subsequently carried out as described in Gibon at al. (2002) Plant J 30: 221-235.

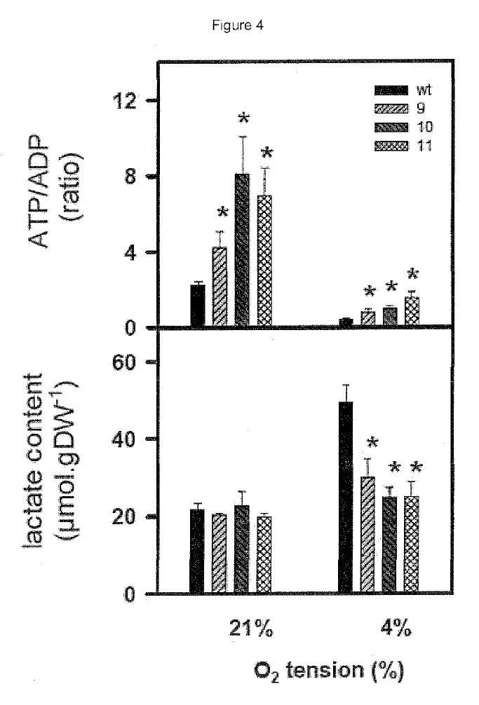

FIG. 4 shows the effect of the seed-specific expression of AHB2 on the ATP/ADP ratio (A) and the lactate content (B) in maturing seeds which had been grown under normal oxygen conditions (21%) or under hypoxic conditions (4%). The results are means and standard deviations from 6 independent measurements. Significant differences to the wild type (based on the statistic t-test analysis) are identified by asterisks (*).

Under natural oxygen concentrations in the environment, the seed-specific overexpression of AHB2 leads to an ATP:ADP ratio which is 2 to 4 times higher in the seeds of the transgenic lines (4-8) than in the wild-type seeds (2). This indicates an improved energy supply by the respiratory chain in transgenic seeds, even under the low oxygen concentrations within the seed.

Lowering the oxygen concentration in the environment to 4% leads, in the wild-type seeds, to a reduced energy status, which is reflected in the reduction of the ATP:ADP ratio from 2 to 0.4. Lowering the energy status was accompanied by the accumulation of lactate in the seeds (20 μmol gDW⁻¹ at 21% O₂; 50 μmol gDW⁻¹). This demonstrates that the wild-type seeds partially compensate for lacking energy by anaerobic fermentation, which is energetically less advantageous.

In the AHB2 overexpressing seeds, lowering the oxygen concentration in the environment to 4% likewise leads to a reduced energy status. However, the ATP:ADP ratio in these plants is 0.8-2 and therefore significantly higher than in the wild-type seeds (0.4). This indicates a continued sufficient aerobic energy supply at an oxygen concentration in the environment of 4%. This finding is confirmed by the fact that the transgenic seeds do not reveal an increase of lactate, which is formed by aerobic fermentation.

1. A method of modifying the ATP/ADP ratio in at least one cell, tissue, organ, microorganism or plant, comprising modifying the activity of at least one hemoprotein.

2. The method according to claim 1, wherein the activity of at least one leghemoglobin is modified.

3. The method according to claim 1, wherein the activity of a hemoprotein is increased.

4. The method according to claim 1, wherein the activity of at least one polypeptide is increased which is encoded by a nucleic acid molecule comprising at least one nucleic acid molecule selected from the group consisting of:
a) a nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by using the nucleic acid molecule as shown in (a) to (c) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

5. The method according to any of claim 1, wherein the activity of at least one hemoprotein is increased by expression, preferably overexpression, which is encoded by a nucleic acid molecule comprising at least one nucleic acid molecule selected from the group consisting of
a) a nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by using a the nucleic acid molecule as shown in (a) to (c) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid
sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

6. The method according to any of claim 1, wherein the leghemoglobin and hemoglobin are selected from plants of the group consisting of Arabidopsis thaliana, Lupinus luteus, Glycine max, Medicago sativa, Medicago trunculata, Phaseolus vulgaris, Vicia faba, Pisum sativum, Vigna unguiculata, Lotus japonicus, Psophocarpus tetragonolobus, Sesbania rostrata, Casuarina glauca and Convallaria lineata.

7. The method according to claim 1, wherein the hemoprotein is from Lotus japonicus or preferably Arabidopsis thaliana.

8. The method according to claim 1, wherein the plants are transformed such that they express the hemoprotein in a storage-organ-specific manner.

9. The method according to claim 1, wherein the plants are transformed such that they express the hemoprotein in a tuber-specific and/or seed-specific manner.

10. The method according to claim 1, wherein monocotyledonous crop plants, in particular of the family Gramineae, are transformed.

11. The method according to claim 1, wherein dicotyledonous crop plants, in particular from the family Asteraceae, Brassicacea, Compositae, Cruciferae, Cucurbitaceae, Leguminosae, Rubiaceae, Solanaceae, Sterculiaceae, Theaceae or Umbelliferae are transformed.

12. The method according to claim 1, wherein potatoes, Arabidopsis thaliana, soybeans or oilseed rape are transformed.

13. A method for the preparation of a polypeptide with hemoprotein activity in at least one cell, tissue, organ, microorganism or plant, comprising transforming a nucleic acid molecule into said cell, tissue, organ, microorganism or plant, wherein the nucleic acid molecule comprises comprising at least one nucleic acid molecule selected from the group consisting of:
a) a nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by means of a the nucleic acid molecule as shown in (a) to (c) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid
sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

14. A method for modifying the ATP/ADP ratio in at least one cell, tissue, organ, microorganism or plant, comprising transforming a nucleic acid molecule into said cell, tissue, organ, microorganism or plant, wherein the nucleic acid molecule comprises at least one nucleic acid molecule selected from the group consisting of:
a) a nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by using a the nucleic acid molecule as shown in (a) to (c) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

15. A method for preparing at least one cell, tissue, organ, microorganism or plant with a modified ATP/ADP ratio, preferably an increased ATP/ADP ratio, comprising transforming a nucleic acid molecule into said cell, tissue, organ, microorganism or plant, wherein the nucleic acid molecule comprises at least one nucleic acid molecule selected from the group consisting of:
a) a nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by using a the nucleic acid molecule as shown in (a) to (e) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid sequence in accordance with the consensus sequence of the hemoprotein sequences which comprises SEQ ID NO 46 and/or 47, preferably SEQ ID NO 43 and/or 44, especially preferably SEQ ID NO 43 and/or 45.

16. A method for preparing at least one cell, tissue, organ, microorganism or plant with a modified oil content, preferably an increased fatty acid content, preferably an increased linolenic acid content, comprising transforming a nucleic acid molecule into said cell, tissue, organ, microorganism or plant, wherein the nucleic acid molecule comprises comprising at least one nucleic acid molecule selected from the group consisting of:
a) a nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by using a the nucleic acid molecule as shown in (a) to (c) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

17. A nucleic acid molecule comprising a nucleic acid molecule selected from the group consisting of:
a) a nucleic acid molecule which codes for a polypeptide comprising the sequence shown in SEQ ID NO 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by using a the nucleic acid molecule as shown in (a) to (e) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45.

18. A nucleic acid molecule comprising a nucleotide sequence which differs in one, two, three, four, five, six, seven, eight, nine, ten or more nucleotides from a nucleic acid molecule selected from the group consisting of
a) a nucleic acid molecule which codes for a polypeptide comprising the sequence shown in SEQ ID NO 2, 4, 6, 8, 10, 12, 14, 16, 18, 20, 22, 32, 33, 34, 35, 36, 37, 38, 39 and/or 40;
b) a nucleic acid molecule which comprises at least one polynucleotide of the sequence shown in SEQ ID NO 1, 3, 5, 7, 9, 11, 13, 15, 17, 19, 21, 23, 24, 25, 26, 27, 28, 29, 30 and/or 31;
c) a nucleic acid molecule which codes for a polypeptide whose sequence has at least 40% identity with the sequences SEQ ID NO 2, 4, 6, 8, 10, 12, 14, 16, 18, 20, 22, 32, 33, 34, 35, 36, 37, 38, 39 and/or 40;
d) a nucleic acid molecule according to (a) to (c) which codes for a fragment of the sequences as shown in SEQ ID NO 2, 4, 6, 8, 10, 12, 14, 16, 18, 20, 22, 32, 33, 34, 35, 36, 37, 38, 39 and/or 40;
e) a nucleic acid molecule which is obtained by amplifying a nucleic acid molecule from a cDNA database or from a genome database by means of the primers as shown in sequence No. 41 and 42;
f) a nucleic acid molecule which codes for a polypeptide with hemoprotein activity and which hybridizes under stringent conditions with a the nucleic acid molecule as shown in (a) to (c);
g) a nucleic acid molecule coding for a hemoprotein which can be isolated from a DNA library under stringent hybridization conditions by using a the nucleic acid molecule as shown in (a) to (c) or the subfragments thereof of at least 15 nt, preferably 20 nt, 30 nt, 50 nt, 100 nt, 200 nt or 500 nt, as the probe; and
h) a nucleic acid molecule coding for a polypeptide comprising an amino acid sequence in accordance with the consensus sequence of the hemoprotein sequences, which comprises SEQ ID NO 46 and/or 47, preferably SEQ ID NO 43 and/or 44, especially preferably SEQ ID NO 43 and/or 45; and which codes for a polypeptide with the activity of a hemoprotein.

19. A protein encoded by the nucleic acid molecule according to claim 17, wherein the protein does not consist of the sequence shown in SEQ ID NO 2 and 4.

20. A DNA expression cassette comprising a nucleic acid sequence which is essentially identical to the nucleic acid molecule according to claim 17 and which codes for a protein that does not consist of the sequence shown in SEQ ID NO 2 and 4.

21. A vector comprising the expression cassette according to claim 20.

22. A transgenic cell comprising the expression cassette according to claim 20 or a vector comprising said expression cassette.


SEQUENCE LISTING (48 sequences)

SEQ ID NO: 1 - DNA, 477 bp, Arabidopsis thaliana
  CDS (1)..(477)
atgggagagattgggtttacagagaagcaagaagctttggtgaaggaatcgtgggagatactgaaacaagacatccccaaatacagccttcacttcttctcacagatactggagatagcaccagcagcaaaaggcttgttctctttcctaagagactcagatgaagtccctcacaacaatcctaaactcaaagctcatgctgttaaagtcttcaagatgacatgtgaaacagctatacagctgagggaggaaggaaaggtggtagtggctgacacaaccctccaatatttaggctcaattcatctcaaaagcggcgttattgaccctcacttcgaggtggtgaaagaagctttgctaaggacattgaaagaggggttgggggagaaatacaatgaagaagtggaaggtgcttggtctcaagcttatgatcacttggctttagccatcaagaccgagatgaaacaagaagagtcataa

SEQ ID NO: 2 - protein, 158 aa, Arabidopsis thaliana
MGEIGFTEKQEALVKESWEILKQDIPKYSLHFFSQILEIAPAAKGLFSFLRDSDEVPHNNPKLKAHAVKVFKMTCETAIQLREEGKVVVADTTLQYLGSIHLKSGVIDPHFEVVKEALLRTLKEGLGEKYNEEVEGAWSQAYDHLALAIKTEMKQEES

SEQ ID NO: 3 - DNA, 444 bp, Lotus japonicus
  CDS (1)..(444)
atgggtttcactgcgcagcaagaggctctagtgggtagctcatacgaaacattcaagaaaaaccttcctaccaacagtgttttgttctacaccgttatattggagatagcaccaactgcaaaagacatgttctcctttctaaaggagtctgggcctaagcatagtcctcagctccaggcccatgctgaaaaggtttttgcactgactcgtgatgctgccactcaactcgtagcaaaaggagaagtgacacttgcagatgccagcttaggtgctgtccacgttcagaaagccgttactgatcctcatttcgtggtggttaaagaagccctgcttcaaacagtaaaggaagcagttggggcggacgaatggagtgatgacttgagcaccgcttgggaaggagcatatgatggactagcaactgcaattaagaaggcaatgggttaa

SEQ ID NO: 4 - protein, 147 aa, Lotus japonicus
MGFTAQQEALVGSSYETFKKNLPTNSVLFYTVILEIAPTAKDMFSFLKESGPKHSPQLQAHAEKVFALTRDAATQLVAKGEVTLADASLGAVHVQKAVTDPHFVVVKEALLQTVKEAVGADEWSDDLSTAWEGAYDGLATAIKKAMG

SEQ ID NO: 5 - DNA, 486 bp, Brassica napus
  CDS (1)..(486)
atgggaaagattgtgtttacagagaagcaagaagctttggtgaaggagtcttgggagatactcaagcaagacatccccaaatacagtcttcacttcttctcacagatactggagatagcaccagcagcgaaggacatgttctctttcctaagagacacagatgaagtccctcataacaatcccaaactcaaagctcatgctgttaaagtcttcaagatgacatgtgaaacagcaatacagctgagggagaaaggaaaagtagtggtggctgacacaaccctccaatacttgggctctgttcatctcaagagcggtgttcttgatcctcactttgaggtggtgaaagaggctttggtgaggacactgaaagaagggttgggggagaagtacaatgaagaagtggaaggagcttggtctcaagcttatgatcacttggctttagccattaaggccgagatgaaacaagaagactcacaaaaaccctaa

SEQ ID NO: 6 - protein, 161 aa, Brassica napus
MGKIVFTEKQEALVKESWEILKQDIPKYSLHFFSQILEIAPAAKDMFSFLRDTDEVPHNNPKLKAHAVKVFKMTCETAIQLREKGKVVVADTTLQYLGSVHLKSGVLDPHFEVVKEALVRTLKEGLGEKYNEEVEGAWSQAYDHLALAIKAEMKQEDSQKP

SEQ ID NO: 7 - DNA, 414 bp, Brassica napus
  CDS (1)..(414)
  misc_feature (358)..(358): n can be a, c, g or t
atggagagtgagggaaagattgtgtttacagaagagcaagaggctcttgtggtgaagtcatggagtgtcatgaagaaaaattcagctgatttgggtctcaaactcttcatcaagatctttgagattgcaccaacagcgaagaagttgttctcctttttgagagactcaccaatccctgctgagcaaaacccaaagctcaagcctcatgccatgtctgtttttgtcatgtgttgtgagtcagcagcacagctgagaaaaacaggaaaagtcacagtgaaggagacaactttgaagaggctaggagccagtcattctaaatacggtgtggttgatgaacactttgaggtgaccaagtatngcattgttggagacaataaaggaggcggtgccagagatgtggtcaccggaaatgaa

SEQ ID NO: 8 - protein, 138 aa, Brassica napus
  misc_feature (120)..(120): The ′Xaa′ at location 120 stands for Ser, Gly, Arg, or Cys.
MESEGKIVFTEEQEALVVKSWSVMKKNSADLGLKLFIKIFEIAPTAKKLFSFLRDSPIPAEQNPKLKPHAMSVFVMCCESAAQLRKTGKVTVKETTLKRLGASHSKYGVVDEHFEVTKYXIVGDNKGGGARDVVTGNE

SEQ ID NO: 9 - DNA, 390 bp, Glycine max
  CDS (1)..(390)
tcattcgaagcattcaaggcaaacattcctcaatacagcgttgtgttctacacttcgatactggagaaagcacctgcagcaaaggacttgttctcatttctagcaaatggagtagaccccactaatcctaagctcacgggccatgctgaaaagctttttgcattggtgcgtgactcagctggtcaacttaaagcaagtggaacagtggtggctgatgccgcacttggttctatccatgcccaaaaagcagtcactgatcctcagttcgtggtggttaaagaagcactgctgaaaacaataaaggaagcagttggggacaaatggagtgacgagttgagcagtgcttgggaagtagcctatgatgaattggcagcagctattaagaaggca

SEQ ID NO: 10 - protein, 130 aa, Glycine max
SFEAFKANIPQYSVVFYTSILEKAPAAKDLFSFLANGVDPTNPKLTGHAEKLFALVRDSAGQLKASGTVVADAALGSIHAQKAVTDPQFVVVKEALLKTIKEAVGDKWSDELSSAWEVAYDELAAAIKKA

SEQ ID NO: 11 - DNA, 471 bp, Glycine max
  CDS (1)..(471)
  misc_feature (395)..(395)
  misc_feature (395)..(395): n is a, c, g, or t
atgaccaccacattggaaagaggtttctcggaagagcaagaagctctggtggtgaagtcatggaatgtcatgaagaagaattctggagagttgggtctcaagtttttcttgaaaatatttgagattgctccatcagctcagaaattgttctcattcttgagagattcaacggttcctttggagcaaaatcccaagctcaagccccatgccgtgtctgtctttgtaatgacctgtgattcagcagttcagctgcggaaggccgggaaagtcactgtcagagaatcaaacttgaaaaaattaggtgctacccattttagaaccggcgtagcaaacgagcatttcgaggtgacaaagtttgcactgttggagaccataaaagaagctgtaccagaaantgtggtcaccggctatgaagaatgcatgggagaagcttatgatcagctggtcgatgccattaaatctgaaatgaaa

SEQ ID NO: 12 - protein, 157 aa, Glycine max
  misc_feature (132)..(132): The ′Xaa′ at location 132 stands for Asn, Ser, Thr, or Ile.
MTTTLERGFSEEQEALVVKSWNVMKKNSGELGLKFFLKIFEIAPSAQKLFSFLRDSTVPLEQNPKLKPHAVSVFVMTCDSAVQLRKAGKVTVRESNLKKLGATHFRTGVANEHFEVTKFALLETIKEAVPEXVVTGYEECMGEAYDQLVDAIKSEMK

SEQ ID NO: 13 - DNA, 624 bp, Oryza sativa
  CDS (1)..(624)
  misc_feature (368)..(368): n can be a, c, g or t
atggctctcgtggagggaaacaacggcgtgtcggggggagcggtcagcttcagcgaggagcaggaggcgcttgtgctcaagtcgtgggccatcatgaagaaggattccgccaacattggactccgcttcttcttgaagatcttcgaggtcgcgccgtcggcgagccagatgttctcgttcctgcgcaactccgacgtgccgctcgagaagaaccccaagctcaagacccacgccatgtccgtcttcgtcatgacatgtgaggccgccgcgcagctgcggaaagccgggaaggtcaccgtgagagacaccaccctgaagaggctcggcgccacgcacttcaagtacggcgtcggagacgcccacttcgnaggtacagtgatccccaatggctgcctgcgctccattcngatcgacatgaaacttgatcgttttctgatcgtgtctttgtcgaacaacgtacatgcgatcgatcgatcgtgtaaacaggtggtgaagttcgcgctgcttgacacgatcaaggaggaggttccggcggacantgtggagcccggcgatgaagagcgcgtggacgaagcctacgaccacctggtcgctgccatcaagcaggagatgaagcccgcggag

SEQ ID NO: 14 - protein, 208 aa, Oryza sativa
  misc_feature (123)..(123): The ′Xaa′ at location 123 stands for Glu, Gly, Ala, or Val.
MALVEGNNGVSGGAVSFSEEQEALVLKSWAIMKKDSANIGLRFFLKIFEVAPSASQMFSFLRNSDVPLEKNPKLKTHAMSVFVMTCEAAAQLRKAGKVTVRDTTLKRLGATHFKYGVGDAHFXGTVIPNGCLRSIXIDMKLDRFLIVSLSNNVHAIDRSCKQVVKFALLDTIKEEVPADXVEPGDEERVDEAYDHLVAAIKQEMKPAE

SEQ ID NO: 15 - DNA, 576 bp, Oryza sativa
  CDS (1)..(576)
atggcactcgcggaggccgacgacggcgcggtggtcttcggcgaggagcaggaggcgctggtgctcaagtcgtgggccgtcatgaagaaggacgccgccaacctgggcctccgcttctttctcaaggtcttcgagatcgcgccgtcggcgaagcagatgttctcgttcctgcgcgactccgacgtgccgctggagaagaaccccaagctcaagacgcacgccatgtccgtcttcgtcatgacctgcgaggcggcggcgcagcttcgcaaggccgggaaggtcaccgtgagggagaccacgctcaagaggctgggcgccacgcacttgaggtacggcgtcgcagatggacacttcgaggtgacggggttcgcgctgcttgagacgatcaaggaggcgctccccgctgacatgtggagcctcgagatgaagaaagcctgggccgaggcctacagccagctggtggcggccatcaagcgggagatgaagcccgatgccagtagtggcgattgcgaccagtgtttaacccatgacgcagcgccgtcacagatgtcccgtgtggtcttgcgctttagcaat

SEQ ID NO: 16 - protein, 192 aa, Oryza sativa
MALAEADDGAVVFGEEQEALVLKSWAVMKKDAANLGLRFFLKVFEIAPSAKQMFSFLRDSDVPLEKNPKLKTHAMSVFVMTCEAAAQLRKAGKVTVRETTLKRLGATHLRYGVADGHFEVTGFALLETIKEALPADMWSLEMKKAWAEAYSQLVAAIKREMKPDASSGDCDQCLTHDAAPSQMSRVVLRFSN

SEQ ID NO: 17 - DNA, 342 bp, Triticum aestivum
  CDS (1)..(342)
atgtccgccgcggagggagccgtcgtgttcagcgaggagaaggaggcgctggtgctcaagtcatgggccatcatgaagaaggattccgccaacctcgggctccgcttcttcctcaagatctttgagatcgcgccgtcgggagagcagatgttcccgttcctgcgcgactccgacgtgccgctggagaccaaccccaagctcaagacccacgccgtgtccgtcttcgtcatgacgtgcgaggcggctgcgcacgtgcggaaagccgggaagatcacggtgagggagaccaccctgaagaggctgggcggcacgcacttgaaatacggcgtggcagatggccac

SEQ ID NO: 18 - protein, 114 aa, Triticum aestivum
MSAAEGAVVFSEEKEALVLKSWAIMKKDSANLGLRFFLKIFEIAPSGEQMFPFLRDSDVPLETNPKLKTHAVSVFVMTCEAAAHVRKAGKITVRETTLKRLGGTHLKYGVADGH

SEQ ID NO: 19 - DNA, 576 bp, Zea mays; identical to SEQ ID NO: 15
  CDS (1)..(576)

SEQ ID NO: 20 - protein, 192 aa, Zea mays; identical to SEQ ID NO: 16

SEQ ID NO: 21 - DNA, 441 bp, Lotus japonicus
  CDS (1)..(441)
atgggtttcactgcacagcaagatgctttagtgggtagctcatatgaagcattcaagcaaaaccttcctagcaatagtgttctgttctacaccttaatattggaaaaagccccagctgctaaagacatgttctcctttctaaaggcttctggacccacgcacagtcctcaactccaagcccatgctgaaaaggtttttggactgacacgcgatgcggctgctcaactcttagcaaaaggagaagtgacacttgcagatgccggcttaggtgctgttcatgttcagaaagcagttgctgacccccatttcgcggtggttaaagaagcactgcttaaaacagtacaggcagcggttggggacaaatggagtgaggatttgagcactgcttggggagtagcttatgatggactcgcagctgcaattaagaaggcaatgagttga

SEQ ID NO: 22 - protein, 146 aa, Lotus japonicus
MGFTAQQDALVGSSYEAFKQNLPSNSVLFYTLILEKAPAAKDMFSFLKASGPTHSPQLQAHAEKVFGLTRDAAAQLLAKGEVTLADAGLGAVHVQKAVADPHFAVVKEALLKTVQAAVGDKWSEDLSTAWGVAYDGLAAAIKKAMS

SEQ ID NO: 23 - DNA, 650 bp, Brassica napus
cagagagctcacaagagagagagctcagagagagagagctcataagagagagatgggaaagattgtgtttacagagaagcaagaagctttggtgaaggagtcttgggagatactcaagcaagacatccccaaatacagtcttcacttcttctcacagatactggagatagcaccagcagcgaaggacatgttctctttcctaagagacacagatgaagtccctcataacaatcccaaactcaaagctcatgctgttaaagtcttcaagatgacatgtgaaacagcaatacagctgagggagaaaggaaaagtagtggtggctgacacaaccctccaatacttgggctctgttcatctcaagagcggtgttcttgatcctcactttgaggtggtgaaagaggctttggtgaggacactgaaagaagggttgggggagaagtacaatgaagaagtggaaggagcttggtctcaagcttatgatcacttggctttagccattaaggccgagatgaaacaagaagactcacaaaaaccctaaatatcatttgggtattatatcattatatgatcatacatatctgtgtatgtagtatacttcatactttgactttctacagttcattgttttttgaataaggattttgagatat

SEQ ID NO: 24 - DNA, 426 bp, Brassica napus
  misc_feature (370)..(370): n can be a, c, g or t
gcctctgaggatatggagagtgagggaaagattgtgtttacagaagagcaagaggctcttgtggtgaagtcatggagtgtcatgaagaaaaattcagctgatttgggtctcaaactcttcatcaagatctttgagattgcaccaacagcgaagaagttgttctcctttttgagagactcaccaatccctgctgagcaaaacccaaagctcaagcctcatgccatgtctgtttttgtcatgtgttgtgagtcagcagcacagctgagaaaaacaggaaaagtcacagtgaaggagacaactttgaagaggctaggagccagtcattctaaatacggtgtggttgatgaacactttgaggtgaccaagtatngcattgttggagacaataaaggaggcggtgccagagatgtggtcaccggaaatgaa

SEQ ID NO: 25 - DNA, 390 bp, Glycine max; identical to SEQ ID NO: 9

SEQ ID NO: 26 - DNA, 483 bp, Glycine max
  misc_feature (407)..(407): n can be a, c, g or t
ctactagacaacatgaccaccacattggaaagaggtttctcggaagagcaagaagctctggtggtgaagtcatggaatgtcatgaagaagaattctggagagttgggtctcaagtttttcttgaaaatatttgagattgctccatcagctcagaaattgttctcattcttgagagattcaacggttcctttggagcaaaatcccaagctcaagccccatgccgtgtctgtctttgtaatgacctgtgattcagcagttcagctgcggaaggccgggaaagtcactgtcagagaatcaaacttgaaaaaattaggtgctacccattttagaaccggcgtagcaaacgagcatttcgaggtgacaaagtttgcactgttggagaccataaaagaagctgtaccagaaantgtggtcaccggctatgaagaatgcatgggagaagcttatgatcagctggtcgatgccattaaatctgaaatgaaa

SEQ ID NO: 27 - DNA, 666 bp, Oryza sativa
  misc_feature (410)..(410): n can be a, c, g or t
tttcgtcgattcaccacacagaggaatcaaatcgaagcagccatggctctcgtggagggaaacaacggcgtgtcggggggagcggtcagcttcagcgaggagcaggaggcgcttgtgctcaagtcgtgggccatcatgaagaaggattccgccaacattggactccgcttcttcttgaagatcttcgaggtcgcgccgtcggcgagccagatgttctcgttcctgcgcaactccgacgtgccgctcgagaagaaccccaagctcaagacccacgccatgtccgtcttcgtcatgacatgtgaggccgccgcgcagctgcggaaagccgggaaggtcaccgtgagagacaccaccctgaagaggctcggcgccacgcacttcaagtacggcgtcggagacgcccacttcgnaggtacagtgatccccaatggctgcctgcgctccattcngatcgacatgaaacttgatcgttttctgatcgtgtctttgtcgaacaacgtacatgcgatcgatcgatcgtgtaaacaggtggtgaagttcgcgctgcttgacacgatcaaggaggaggttccggcggacantgtggagcccggcgatgaagagcgcgtggacgaagcctacgaccacctggtcgctgccatcaagcaggagatgaagcccgcggag

SEQ ID NO: 28 - DNA, 442 bp, Oryza sativa
tcgacgatttcgtcggaagagcaagaagctcggtggtgaaatcatggaacgaaatgaagaagaattctcaagaactcggtctaaattttttcaagaaaatattggagattgctccagcagctcagcaattgttctcattcttgaaggattcaacgttcctttggaggaaaaccccaagctcaaaccccatgccatggctgtctttgtcatgacgtgtgaatcagctgttcaactgaggaaagctggtaaagtcactgtgagggaatcaaacttgaaaagattaggtgctactcattttaaagccggtgtagcagctgagcatttcgaggtaacaaagttggcactattggagaccataaaagaagcagtgcctgaaatgtggtcaccagccatgaagaatgcatgggaagaagctcatgatcagctggccgaagccatcaaa

SEQ ID NO: 29 - DNA, 354 bp, Triticum aestivum
agggaggaagccatgtccgccgcggagggagccgtcgtgttcagcgaggagaaggaggcgctggtgctcaagtcatgggccatcatgaagaaggattccgccaacctcgggctccgcttcttcctcaagatctttgagatcgcgccgtcgggagagcagatgttcccgttcctgcgcgactccgacgtgccgctggagaccaaccccaagctcaagacccacgccgtgtccgtcttcgtcatgacgtgcgaggcggctgcgcacgtgcggaaagccgggaagatcacggtgagggagaccaccctgaagaggctgggcggcacgcacttgaaatacggcgtggcagatggccac

SEQ ID NO: 30 - DNA, 663 bp, Zea mays
atcccaccagtgtccagtgctcggggaaccgacacagctcctcagcagagtagccagcacgacaagcccgatcagcagacagcaggcatggcactcgcggaggccgacgacggcgcggtggtcttcggcgaggagcaggaggcgctggtgctcaagtcgtgggccgtcatgaagaaggacgccgccaacctgggcctccgcttctttctcaaggtcttcgagatcgcgccgtcggcgaagcagatgttctcgttcctgcgcgactccgacgtgccgctggagaagaaccccaagctcaagacgcacgccatgtccgtcttcgtcatgacctgcgaggcggcggcgcagcttcgcaaggccgggaaggtcaccgtgagggagaccacgctcaagaggctgggcgccacgcacttgaggtacggcgtcgcagatggacacttcgaggtgacggggttcgcgctgcttgagacgatcaaggaggcgctccccgctgacatgtggagcctcgagatgaagaaagcctgggccgaggcctacagccagctggtggcggccatcaagcgggagatgaagcccgatgccagtagtggcgattgcgaccagtgtttaacccatgacgcagcgccgtcacagatgtcccgtgtggtcttgcgctttagcaat

SEQ ID NO: 31 - DNA, 441 bp, Lotus japonicus; identical to SEQ ID NO: 21

SEQ ID NO: 32 - protein, 161 aa, Brassica napus; identical to SEQ ID NO: 6

SEQ ID NO: 33 - protein, 138 aa, Brassica napus; identical to SEQ ID NO: 8
  MISC_FEATURE (120)..(120): Xaa can be any amino acid

SEQ ID NO: 34 - protein, 130 aa, Glycine max; identical to SEQ ID NO: 10

SEQ ID NO: 35 - protein, 157 aa, Glycine max; identical to SEQ ID NO: 12
  MISC_FEATURE (132)..(132): Xaa can be any amino acid

SEQ ID NO: 36 - protein, 208 aa, Oryza sativa; identical to SEQ ID NO: 14
  MISC_FEATURE (123)..(123): Xaa can be any amino acid

SEQ ID NO: 37 - protein, 147 aa, Oryza sativa
RRFRRKSKKLGGEIMERNEEEFSRTRSKFFQENIGDCSSSSAIVLILEGFNVPLEENPKLKPHAMAVFVMTCESAVQLRKAGKVTVRESNLKRLGATHFKAGVAAEHFEVTKLALLETIKEAVPEMWSPAMKNAWEEAHDQLAEAIK

SEQ ID NO: 38 - protein, 114 aa, Triticum aestivum; identical to SEQ ID NO: 18

SEQ ID NO: 39 - protein, 192 aa, Zea mays; identical to SEQ ID NO: 16

SEQ ID NO: 40 - protein, 146 aa, Lotus japonicus; identical to SEQ ID NO: 22

SEQ ID NO: 41 - DNA, 32 bp, Artificial sequence
  primer
tttggtaccatggggagattgggtttacagag

SEQ ID NO: 42 - DNA, 35 bp, Artificial sequence
  primer
tttggatccttatgacctttcttgtttcatctcgg

SEQ ID NO: 43 - protein, 37 aa, Artificial sequence
  consensus
KXAXXXTXKXXXXXXXXXXXXXXXXXXAXXXLXXAIK

SEQ ID NO: 44 - protein, 47 aa, Artificial sequence
  consensus
NPKLKXHAXXXFXXTCXXAXQXXXXGKXTXXXXXLXXLGXXHXXXGV

SEQ ID NO: 45 - protein, 106 aa, Artificial sequence
  consensus
FXXXXXALVXXSXXXXKXXXXXXXXXFXXXXXEXAPXAXXXFSFLXXXXXXXXXNPKLKXHAXXXFXXTCXXAXQXXXXGKXTXXXXXLXXLGXXHXXXGVXXXHF

SEQ ID NO: 46 - protein, 37 aa, Artificial sequence; identical to SEQ ID NO: 43
  consensus

SEQ ID NO: 47 - protein, 47 aa, Artificial sequence; identical to SEQ ID NO: 44
  consensus

SEQ ID NO: 48 - DNA, 33 bp, Artificial sequence
  Primer
tttggtaccatgggagagattgggtttacagag